(19) United States
(12) Patent Application Publication
(10) Pub. No.: US 2014/0039281 A1
(43) Pub. Date: Feb. 6, 2014

(54) GASTRIC BANDING SYSTEM ADJUSTMENT BASED ON A SATIETY AGENT CONCENTRATION LEVEL

(71) Applicant: Allergan, Inc., Irvine, CA (US)

(72) Inventors: Joseph Raven, Tigard, OR (US); Dennis Van Epps, Goleta, CA (US)

(73) Assignee: Allergan, Inc., Irvine, CA (US)

(21) Appl. No.: 14/049,548

(22) Filed: Oct. 9, 2013

Related U.S. Application Data
(63) Continuation of application No. 12/938,755, filed on Nov. 3, 2010, now abandoned.

(51) Int. Cl. A61F 5/00; A61B 5/00; A61B 5/145

(52) U.S. Cl. CPC A61F 5/0053; A61B 5/14546; A61B 5/4848; A61B 5/4836; A61B 5/0004
USPC 600/309; 600/37

(57) ABSTRACT

Methods and devices are provided for adjusting a gastric band to obtain a predetermined optimal expression of hormones. These hormones may be used to control appetite, metabolism and other weight control mechanisms. Ultimately, weight-loss efficacy is desired by tightening or loosening the gastric band based on hormone response.

### CROSS REFERENCE TO RELATED APPLICATION

The present application is a continuation of U.S. patent application Ser. No. 12/938,755, filed Nov. 3, 2010, the disclosure of which is hereby incorporated in its entirety herein by reference.

### FIELD

The present invention generally relates to medical systems, apparatus and uses thereof for treating obesity and/or obesity-related diseases, and more specifically, relates to the adjustment of a gastric banding system based on a satiety agent concentration level.

### BACKGROUND

Adjustable gastric banding systems provide an effective and substantially less invasive alternative to gastric bypass surgery and other conventional surgical weight loss procedures. Despite the sustained weight loss of invasive weight loss procedures, such as gastric bypass surgery, it has been recognized that sustained weight loss can also be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts food's passage into a lower portion of the stomach. When the stoma is of an appropriate size to be partially restricted by a gastric band, the tension on the upper portion of the stomach at rest and during food passage provides a feeling of satiety or fullness, thereby facilitating the reduction of the daily caloric intake. Unlike gastric bypass procedures, the gastric band apparatus are reversible and require no permanent modification to the gastrointestinal tract.

Traditionally gastric bands provide a subcutaneous fluid access port connected to an expandable or inflatable portion of the gastric band. By adding or removing fluid to or from the inflatable portion by means of a hypodermic needle inserted into the access port, the effective size of the band can be adjusted to provide tighter or looser constriction.

Dlugos, et al., U.S. Patent Application Pub. No. 2008/0015406 discloses a pressure-reading device used to measure the pressure. As described, a physician may hold the pressure-reading device against the patient's skin near the location of an injection port and observe the readings. However, using the pressure to determine appropriate stoma size is still lacking as it fails to take into account other important considerations. In other words, such an approach is still too primitive to be an effective stoma-size determining technique.

Recent advances have added automatic adjustable gastric banding systems as well as remotely adjustable gastric banding systems. The following are examples of such systems: Birk, et al., U.S. Patent Application Pub. No. 2010/0010291, herewith incorporated by reference in its entirety, discloses a system for facilitating obesity control which includes an inflatable gastric banding device, a fluid reservoir coupled to the inflatable portion, and an implantable fluid handling device coupled to the fluid reservoir and the inflatable portion. The fluid handling device further includes remotely operable components housed in a biocompatible housing. The fluid handling device also incorporates a micropump effective to pump fluid to the band and a flow sensor.

Birk, U.S. Patent Application Pub. No. 2007/0156013, herewith incorporated in its entirety, discloses an automatically adjustable gastric band system including an adjustment assembly that includes a sensor for sensing fluid pressure in the expandable portion of a gastric band. The adjustment assembly further includes an implantable pump connected to the expandable portion, and a controller for operating the pump to allow for automatic adjustment of the volume of the fluid in the band based on the sensed fluid pressure.

Birk, et al., U.S. Patent Application Pub. No. 2007/0265645 herewith incorporated in its entirety, discloses a self-regulating gastric band adjustment assembly including an implantable fluid reservoir for containing a volume of the fluid useful for adjusting the gastric band.

Coe, U.S. Pat. No. 7,338,433, herewith incorporated in its entirety, discloses a remotely controllable gastric banding system including a pressurized reservoir with valves, and a controller for remotely controlling the valves from outside the patient.

Regardless of how the band is controlled, over time, a stoma created by the gastric band may need an adjustment in order to maintain an appropriate size, which is neither too restrictive nor too passive. However, a simplistic approach to adjust the stoma size based merely on a patient's weight or based on a patient's observations might not be the most effective method of treating obesity.

Recent studies have shown that certain hormones, including “gut” hormones may impact obesity. These gut hormones appear to act as meal initiators and terminators. As such, alterations in the levels of gut hormones may contribute to appetite suppression and sustained weight loss. For example, as noted by Chaudhri, et al., in the paper “Can Gut Hormones Control Appetite and Prevent Obesity?” as published in Diabetes Care, Volume 31, Supplement 2, February 2008, fully incorporated by reference herein, different hormones such as oxyntomodulin, peptide YY, glucagon-like peptide 1, pancreatic polypeptide, and ghrelin may have characteristics as an anti-obesity treatment. Moreover, as confirmed in Cummings, et al., “Gastrointestinal Regulation of Food Intake” as published in the Journal of Clinical Investigation, Volume 117, Number 1, January 2007, herewith incorporated by reference in its entirety, hormones may have beneficial weight loss effects. For example, Cummings notes that glucagon-like peptide-1 (GLP-1), which is expressed in the gut, pancreas, and brain are implicated in satiation.

More particularly, GLP-1 is produced primarily by L cells in the distal small intestine and colon, where it colocalizes with oxyntomodulin and peptide YY (PYY). Ingested nutrients, especially fats and carbohydrates, stimulate GLP-1 secretion by indirect, duodenally activated neurohumoral mechanisms, as well as by direct contact within the distal intestine. Notably, GLP-1 decreases food intake in several species, including humans. Peripheral injections elicit satiety among normal-weight, obese, and diabetic persons. Importantly, patients with diabetes treated with either GLP-1 or the GLP-1 receptor (GLP1R) agonist exenatide lose weight progressively in trials lasting up to two years. This is especially remarkable because improved glycemic control achieved with other agents typically promotes weight gain. Cummings also describes the effects of other hormones on weight loss.

Accordingly, it would be desirable to determine the appropriate size of the stoma by using a gastric band in conjunction with other factors such as increasing hormone levels to achieve more effective weight loss.

### SUMMARY

By using a physiological response (e.g., hormone concentration levels) as opposed to a subjective patient response, increased reliability and sustainability of weight loss may be achieved. For example, by measuring and altering the concentration levels of hormones such as: GLP-1, Peptide YY (PYY), Pancreatic Polypeptide (PP), Oxyntomodulin (OXM), Amylin, Ghrelin, Leptin, Gastrin, Cholecystokinin (CCK), among others, more effective weight loss may be sustained. Indeed, concentration levels of these hormones may be more accurate indicators of a patient's satiety level as compared to, for example, asking the patient how he or she feels, and additionally, may be a better indicator of how tight or loose the gastric band in the patient should be set.

In one embodiment, by changing the concentration level of one or more of these hormones in a patient, weight loss factors such as appetite suppression, metabolism, and the like may be more effectively controlled. In addition, because certain hormones may be produced or reduced with the tightening or loosening of the gastric band, measuring the concentration level of one or more of these hormones may provide a more accurate and effective method of determining how tight or loose the gastric band should be set to achieve the optimal hormone levels for weight loss.

In one embodiment, a method to ascertain whether a gastric band adjustment results in optimal hormone levels for maximum weight loss is provided. First, a patient's gastric band is adjusted. Next, after a predetermined amount of time, a blood sample is obtained from the patient and tested to determine hormone levels. The hormone levels may be compared to pre-determined ranges to ascertain whether the gastric band adjustment results in optimal hormone levels for maximum weight loss. If needed, a further adjustment of the gastric band may be used to increase or decrease the hormone levels closer to the optimal hormone levels.

In another embodiment, another method to ascertain whether a gastric band adjustment results in optimal hormone levels for maximum weight loss is provided. First, an initial blood sample may be obtained from a patient. Next, the patient's gastric band may be adjusted. Following a predetermined period of time, a second blood sample may be obtained. The first and second blood samples may then be tested to determine hormone levels, and the difference therebetween calculated. The difference in hormone levels between the first and second blood samples may be compared against a predetermined range to ascertain whether the gastric band adjustment results in optimal hormone levels for maximal weight-loss. If needed, a further adjustment of the gastric band may be used to increase or decrease the hormone levels closer to the optimal hormone levels.

In another embodiment, an internal device may be used to perform one or more steps related to any of the methods of ascertaining whether a gastric band adjustment results in optimal hormone levels for maximum weight loss as described herein. The internal device may include a sensor for obtaining the hormone levels in the patient, a memory for storing the results, a transceiver for transmitting the results to a computer outside of the patient's body, and a processor for controlling the sensor, the memory and the transceiver.

In one embodiment, the transceiver may be further configured to communicate with the controller of the adjustable gastric band. For example, the transceiver may transmit instructions to tighten or loosen the band, and/or may receive setting levels of the gastric band.

In one embodiment, a method for determining an optimal amount of constriction applied by a gastric band implanted around a stomach of an obese patient is disclosed. The method may include laparoscopically implanting the gastric band around the stomach of an obese patient. The gastric band may have an inflatable portion that can be filled or emptied with fluid (e.g., saline) using an access port to decrease or increase an inner diameter of the gastric band to decrease or increase the size of the opening of the stomach of the obese patient. An obese patient generally has a body mass index (BMI) of 30 or greater. The method may also include collecting a blood, serum or plasma sample from the obese patient and detecting a concentration level of one or more satiety-inducing agents in the sample. If the concentration level is higher than optimal, the gastric band is adjusted (e.g., by removing fluid from the gastric band) so that it applies a lesser constriction to the stomach. If the concentration level is lower than optimal, the gastric band is adjusted (e.g., by adding fluid to the gastric band) so that it applies a greater constriction to the stomach.

### BRIEF DESCRIPTION OF THE DRAWINGS

The following detailed descriptions are given by way of example, but not intended to limit the scope of the disclosure solely to the specific embodiments described herein, may best be understood in conjunction with the accompanying drawings in which:

FIGS. 1A and 1B, respectively, illustrate a perspective view of an implantable gastric band and a side view of the band in accordance with one or more embodiments described herein;

FIG. 2 illustrates a flow chart of a method of determining whether a patient is at or near an optimal hormone concentration level in accordance with one or more embodiments described herein;

FIG. 3 illustrates a flow chart of another method of determining whether a patient is at or near an optimal hormone concentration level in accordance with one or more embodiments described herein;

FIG. 4 illustrates a flow chart of yet another method of determining whether a patient is at or near an optimal hormone concentration level in accordance with one or more embodiments described herein;

FIG. 5 illustrates a block diagram of a sensor used to measure a hormone concentration level in accordance with one or more embodiments described herein;

FIG. 6 is a schematic illustration of one example of a gastric band system used in conjunction with hormone detection in accordance with one or more embodiments described herein; and

FIGS. 7A-7G illustrates a plurality of different hormone response models in accordance with one or more embodiments described herein.

### DETAILED DESCRIPTION

The present disclosure generally provides adjustable gastric banding systems, for example, for treatment of obesity and obesity related conditions, as well as systems for controlling inflation of a gastric banding system based on a measured physiological response to the adjustment of the gastric band.

Persons skilled in the art will readily appreciate that various aspects of the disclosure may be realized by any number of methods and devices configured to perform the intended functions. Stated differently, other methods and devices may be incorporated herein to perform the intended functions. It should also be noted that the drawing FIGS. referred to herein are not all drawn to scale, but may be exaggerated to illustrate various aspects of the invention, and in that regard, the drawing FIGS. should not be construed as limiting. Finally, although the present invention may be described in connection with various medical principles and beliefs, the present invention should not be bound by theory.

By way of example, the present disclosure will reference hydraulically adjustable gastric bands. Nevertheless, persons skilled in the art will readily appreciate that the present disclosure advantageously may be applied to one of the numerous varieties of fluid filled surgical implants presently comprising, or which may in the future comprise, access ports. Similarly, while the present invention will be described primarily with reference to fluid filled surgical implants, persons skilled in the art will readily appreciate that the present invention advantageously may be applied to other devices, and whether fluid or gel filled.

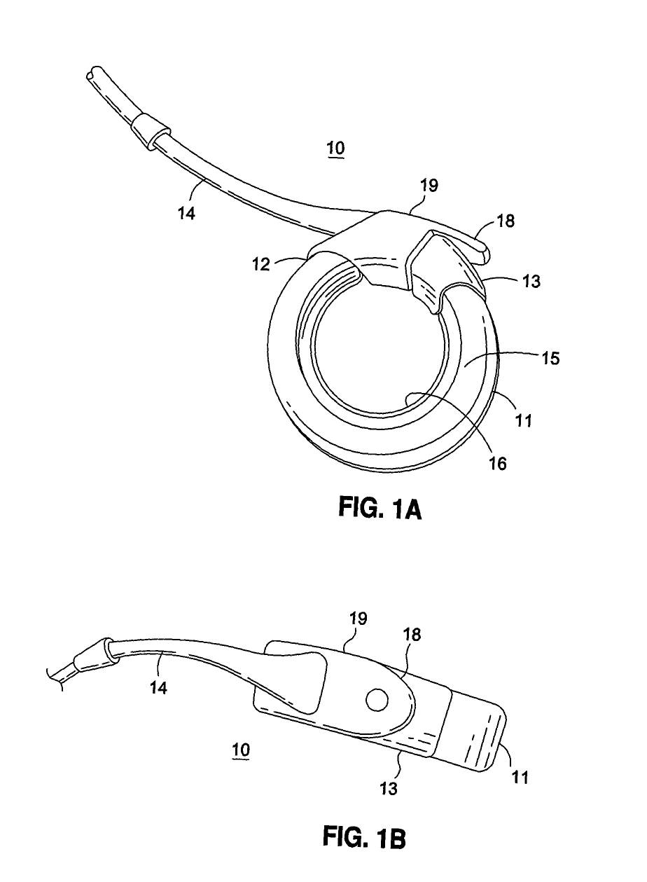

Referring to FIG. 1A, a gastric band 10 in accordance with the present disclosure includes a body portion 11 with an inner stomach-facing surface 15. The body portion 11 has a head end 12 and a tail end or “belt” 13. A fill tube 14, which is generally a tube having a single lumen coextensive therewith, is in fluid communication with an inflatable chamber 16 (also can be referred to as an inflatable member or an inflatable portion) on the inner surface 15 of the band body 11. Preferably, the inflatable chamber 16 is substantially coextensive with the inner surface 15 of the body portion 11. The central lumen of the fill tube 14 is in fluid communication with the inflatable chamber 16. The head end 12 of the body portion 11 has a “buckle” 19 through which the tail end of “belt” 13 is inserted and locked in place in use. The head end 12 may be provided with a pull tab 18 for use in locking the band in place about the stomach.

In use, the gastric band 10 is placed in an encircling position around the stomach and locked in place as shown in FIG. 1B (In FIG. 1B, the stomach is omitted for clarity.). This is accomplished by introducing the gastric band 10 through a laparoscopic cannula (not shown) in a patient's abdominal cavity. The laparoscopic placement includes blunt dissection below the gastro-esophageal junction followed by placement of the gastric band 10. The end of the fill tube 14 is passed through the dissected path around the upper stomach, and the tail end or belt 13 is passed through the buckle 19, so that the belt 13 and the buckle 19 lock in place. A laparoscopic closure tool, such as that disclosed by Coe and Vincent in U.S. Pat. No. 5,658,298, incorporated herein by reference, may be used. Hence, with the gastric band 10 affixed in an encircling position around the stomach, a new stoma (opening) is created within the stomach. After the gastric band 10 is secured in position, the size of the stoma may be adjusted by adding fluid to or withdrawing fluid from the inflatable chamber 16 to bring the stoma opening to the desired size. The inflatable chamber 16 is preferably coextensive with the inner stomach-facing surface 15 of the gastric band 10 between the head end 12 and the tail end 13. The interior of the adjustable chamber 16 is in fluid communication with a fluid reservoir (not shown) by means of the central lumen of the fill tube 14, as with prior art adjustable gastric bands. The inflatable chamber 16 is gradually inflated or deflated with saline or other biologically compatible fluid via the fluid reservoir such that the inflatable chamber 16 presses on and constricts the stomach wall or other tissue underlying the gastric band 10. This results in the decrease or increase of the size of the stomach opening directly inside the encircling gastric band 10.

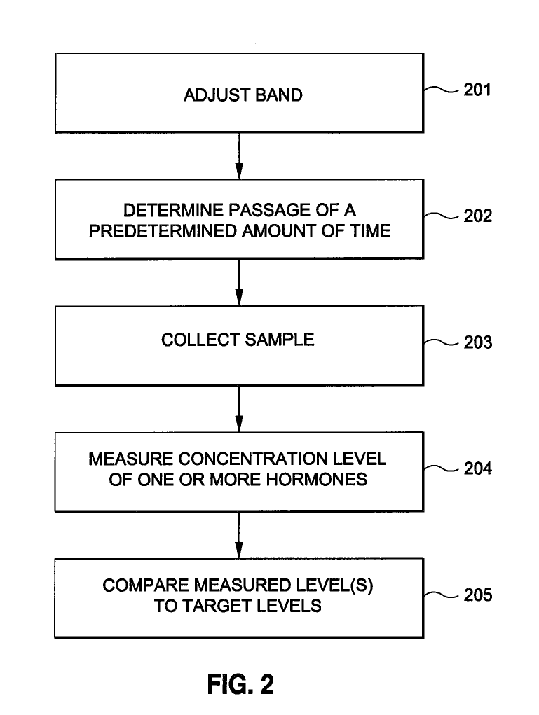

FIG. 2 illustrates a flow chart of a method of determining whether a patient is at or near an optimal hormone concentration level. At step 201, the gastric band (e.g., the gastric band 10 of FIG. 1A) is adjusted to form a tighter constriction about the stoma. In one example, tightening the gastric band about the stoma may be performed by adding fluid to the gastric band. At step 202, a period of time may be observed so that the impact of the tightening of the gastric band may be reflected by the collection of the fluid sample at step 203 (e.g., collecting of a blood sample from the patient). The sample may be obtained using one of a number of traditional fashions such as by pricking the patient's finger, using a needle inserted subcutaneously into the patient's vein, and the like. The amount of blood to be collected may vary from a drop or two to a few cubic centimeters. At step 204, the blood sample may be measured to determine the level of concentration of a particular satiety-inducing agent such as a hormone. At step 205, the measured concentration level of the satiety-inducing agent obtained from the patient's blood may be compared to a pre-determined level. In one embodiment, the pre-determined level of concentration may be based on a combination of the patient's personal data including historical levels previously gathered and general population data such as an average hormone level for a category that the patient is within (e.g., based on sex, ethnicity, height, weight, body mass and the like). The result of the comparison step of 205 may be used to determine whether the tightening of the band in step 201 results in optimal hormone levels.

In one embodiment, the adjustment of the band is performed in conjunction with the patient eating a meal. In this manner, the hormone response to the tightening of the gastric band may indicate whether the patient is still hungry after eating the meal or whether the patient feels full or satiated.

As discussed herein, a predetermined amount of time may be any period of time ranging from 5 minutes to multiple hours (e.g., 15 minutes, 30 minutes, 1 hour, 2 hours and the like). However, in one embodiment, the predetermined time might be the amount of time a particular person takes to release hormones in response to eating food and adjustment of the gastric band. In the average patient, the period of time may be 30 minutes, however, this may be customizably adjustable by a physician to best suit the particular patient. In one embodiment, the hormone response may be monitored continuously during a first visit after a patient consumes a meal/tightening of the gastric band in order to determine the appropriate period of time. In another embodiment, the predetermined amount of time may correlate with the specific hormone the physician is testing for. In one example, GLP-1 may be released about 10-15 minutes after the band is tightened, while oxyntomodulin might not be released until about 90-150 minutes after the tightening.

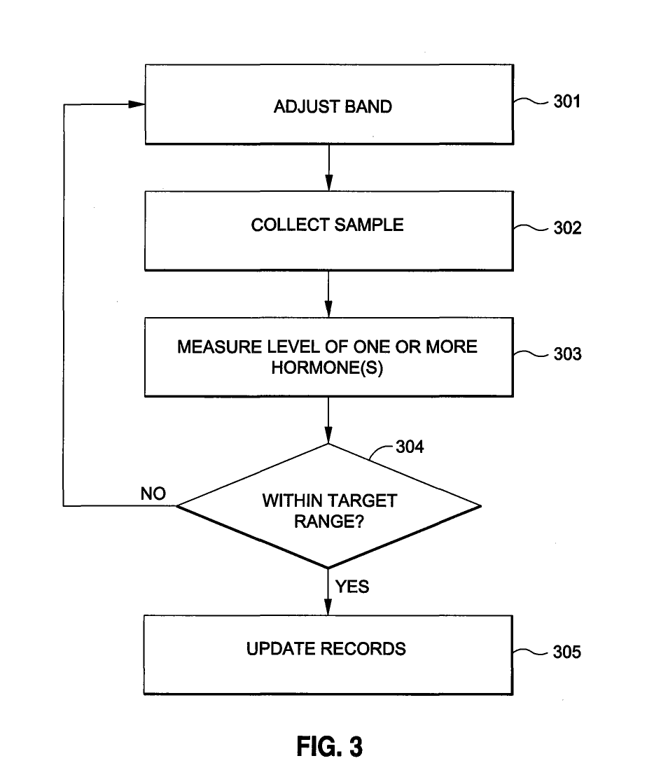

Turning to FIG. 3, a flow chart of a method for determining whether a patient is at or near an optimal hormone concentration level is provided. At step 301, a gastric band attached to a patient is adjusted at the time coincidental to, immediately after, or immediately prior to the patient having a meal. At step 302, a fluid-sample is obtained (e.g., a blood sample) from the patient after the passing of a predetermined period of time. At step 303, the fluid sample may be measured to determine the concentration level of one or more satiety-inducing agents (e.g., hormones). At step 304, a comparison is made to ascertain whether the determined concentration levels are within a target range. If so, the records corresponding to the patient may be updated to detail the adjustment made in step 301 and the resulting concentration level of the one or more satiety inducing agents. However, if the result of the comparison of step 304 lies outside the target range, the process may revert back to step 301 and the gastric band may be re-adjusted. In one embodiment, re-adjustment of the gastric band may be based on the physiological response (e.g., the concentration level of the hormones) alone or the re-adjustment of the band may be based upon a combination of the physiological response and verbal feedback from the patient.

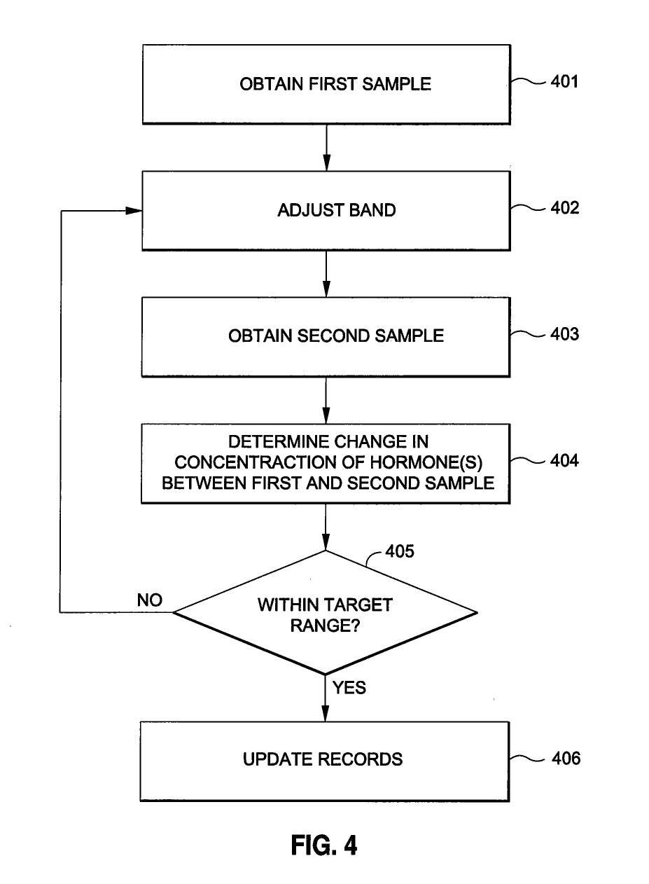

FIG. 4 illustrates a flow chart of another method of determining whether a patient is at or near an optimal hormone concentration level. At step 401, a first fluid sample may be obtained from the patient (e.g., blood sample). At step 402, the gastric band (e.g., gastric band 100) may be adjusted. In one embodiment, the adjustment of the gastric band is performed in conjunction with the patient eating a meal. At step 403, a second fluid sample may be obtained from the patient. At step 404, a difference in a concentration level of one or more satiety-inducing agents between the first sample and the second sample is determined. At step 405, the difference is compared to a target range. If the difference is within the target range, patient records may be updated to reflect hormone levels and the band adjustment details in step 406. However, if the difference is not within the target range, the process may return to step 402, and the gastric band may be re-adjusted. In one embodiment, the process may be repeated until the hormone levels differences are within an acceptable range or until the patient indicates discomfort. Alternatively, re-adjustment of the gastric band may be delayed until the next patient meal.

In another embodiment, for situations where it appears that the patient might not be able to produce the optimal amount of hormones with adjustment of the gastric band for reasons such as discomfort with the tightness of the gastric band or other physiological reasons, the gastric band may be configured to release the hormone in order to raise the patient's hormone level to the optimal hormone level. Methods for configuring the gastric band to release such hormones are disclosed in Raven, U.S. patent application Ser. No. 12/771,671, herewith incorporated in its entirety. In one embodiment, the physician may calculate the deficiency of hormone level and configure the band to release an amount equal to the deficiency.

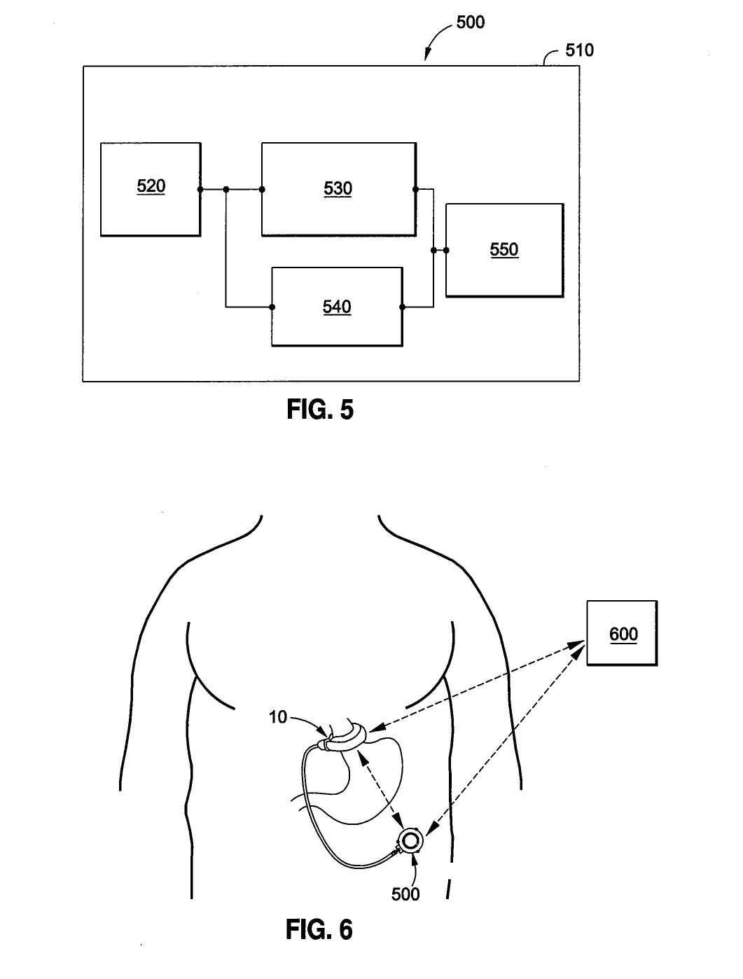

Some embodiments described herein are directed to the scenario where collection of the fluid sample and determination of the hormone level is performed with the assistance of a physician or other medical personal (e.g., in obtaining the blood sample or running a test to determine hormone level). However, utilization of a sensor may replace the need to perform the aforementioned steps by the physician or medical personal. FIG. 5 is a block diagram of one possible sensor which may be used in conjunction with the methods described herein.

FIG. 5 illustrates a hormone-sensing device 500 with a housing 510, a sensor 520 attached to a surface of the device 500 coupled to a processor 530 and a memory 540. The processor 530 and the memory 540 may be coupled to a transceiver 550. The sensor 520 may be configured to determine a concentration level of one or more hormones in the patient and pass along that information for storage in the memory 540. The device 500 may be powered by a battery (not shown) or alternatively via any one of a number of known powering methods. One such method may involve an antenna and/or a rectifier device which receives electromagnetic radiation from an external source and delivers electrical power derived therefrom. A detailed explanation of these and other power delivering methods may be found described in U.S. Pat. No. 6,682,480 issued to Habib et al., herein incorporated by reference. The processor 530 may be configured to instruct the sensor 520 and may further be configured to execute instructions stored in the memory 540. The memory 540 may be a physical memory such as Read-Only Memory (ROM) or any other known storage device. The processor 530 may further be configured to receive or send instructions (and/or other data) to and from the transceiver 550. The transceiver 550 may be configured to transmit and receive signals from a source outside the patient's body, such as a computer, and may be further configured to relay these signals to the processor 530. In one embodiment, the processor 530 may be further configured to instruct the gastric band to release hormones (if necessary).

By utilizing the hormone-sensing device 500, a physician may be able to obtain or collect data from a sample in accordance with one or more embodiment herein without invasively using a needle and/or syringe or other traditional techniques.

In one embodiment, the sensor may be placed remotely from the gastric band system and may communicate wirelessly with the gastric band system and/or an external computer. FIG. 6 depicts a schematic view of a gastric band system in communication with a sensor located in the patient's arm and a computer system external to the patient's body. In this example, the sensor may be a stand-alone device and may be embedded subcutaneously in the patient's body. In one embodiment, the sensor measures the hormone level by analyzing the interstitial fluid. In another embodiment, the sensor may have access to blood within the patient's body and may determine hormone levels and the like by analyzing the concentrations of hormones in the blood.

As shown in FIG. 6, the hormone sensor device 500 may be placed in the upper torso of the patient beneath the skin. The sensor device 500 may, in one embodiment, obtain hormone levels of particular hormones, and may relay that information with the external computer 600. In addition, the sensor 500 may communicate with the gastric band 100 and may instruct the band 100 to either tighten or loosen based on the hormone levels detected.

In another embodiment, the hormone sensing device 500 may be part of the gastric band system, for example, attached to an electronically enhanced access port. In one embodiment, the hormone sensing device 500 of FIG. 5 may be attached to an implanted access port, such as the implanted access port disclosed in U.S. Patent Application No. 61/330,266 (App. '266), herein incorporated by reference in its entirety. For example, the hormone sensing device 500 may be integrated into the access port depicted in FIG. 3B of App. '266. In one embodiment, redundant components may be removed. Alternatively, the entire sensor may be attached to an outside surface of the access port shown in FIG. 1A of App. '266.

As discussed above, the methods described herein may be performed in conjunction with the hormone sensing device 500 described above. For example, referring to FIG. 4, the hormone sensing device 500 of FIG. 5 may be utilized at step 401 to analyze interstitial fluid within a patient to gather hormone concentration levels. At step 402, the gastric band (e.g., gastric band 100) may be adjusted. In one embodiment, the adjustment of the band is performed in conjunction with the patient eating a meal. At step 403, the hormone sensing device 500 of FIG. 5 may be further utilized to re-analyze interstitial fluid within the patient to gather hormone concentration levels after the passage of a predetermined amount of time. At step 404, a difference in a concentration level of one or more satiety-inducing agents between the first sample and the second sample may be determined by the hormone sensing device 500 of FIG. 5. At step 405, the difference is compared to a target range stored in the hormone sensing device 500 of FIG. 5. If the difference is within the target range, the hormone sensing device 500 of FIG. 5 may transmit the relevant data to an external computer and patient records may be updated to reflect hormone levels and the band adjustment details in step 406. However, if the difference is not within the target range, the process may return to step 402, and the hormone sensing device 500 may communicate with the gastric band inside the patient to either tighten or loosen based on the measured hormone level differences. In one embodiment, the process may be repeated until the hormone levels differences are within an acceptable range or until the patient indicates discomfort. Alternatively, re-adjustment of the gastric band may be delayed until the next patient meal. In another embodiment, the external computer may be configured to control the execution of the hormone sensor device 500 of FIG. 5. For example, in the instance where a patient complains about the tightness of the gastric band, the external computer may instruct the sensor to stop communication with the gastric band, or alternatively instruct the sensor to command the gastric band to loosen.

Turning to the satiety-inducing agents (e.g., hormones) discussed herein, one or more of these satiety-inducing agents may be useful in assisting a patient to lose weight. For example, these hormones include: GLP-1, PYY, PP, OXM, Amylin, Ghrelin, Leptin, Gastrin, CCK, among others.

In one embodiment, tightening of the gastric band contemporaneous with the patient eating a meal may have a direct impact on the level of hormone production in the patient. More particularly, tightening of the gastric band (and thus forming a smaller stoma) may trigger a production and/or release of hormones in the patient. However, it should be noted that either tightening the band or eating a meal alone might not result in the desired response of increased hormone production. Similarly, mere tightening of the band and the patient consuming food might also not result in the response of the desired hormone production level. In other words, the magnitude of the tightening of the band may be more important with respect to reaching the optimal level of satiety-inducing hormone production than mere tightening of the gastric band without regard to magnitude. Hence, by using one or more of the methods described herein, improved weight-loss efficacy may be achieved.

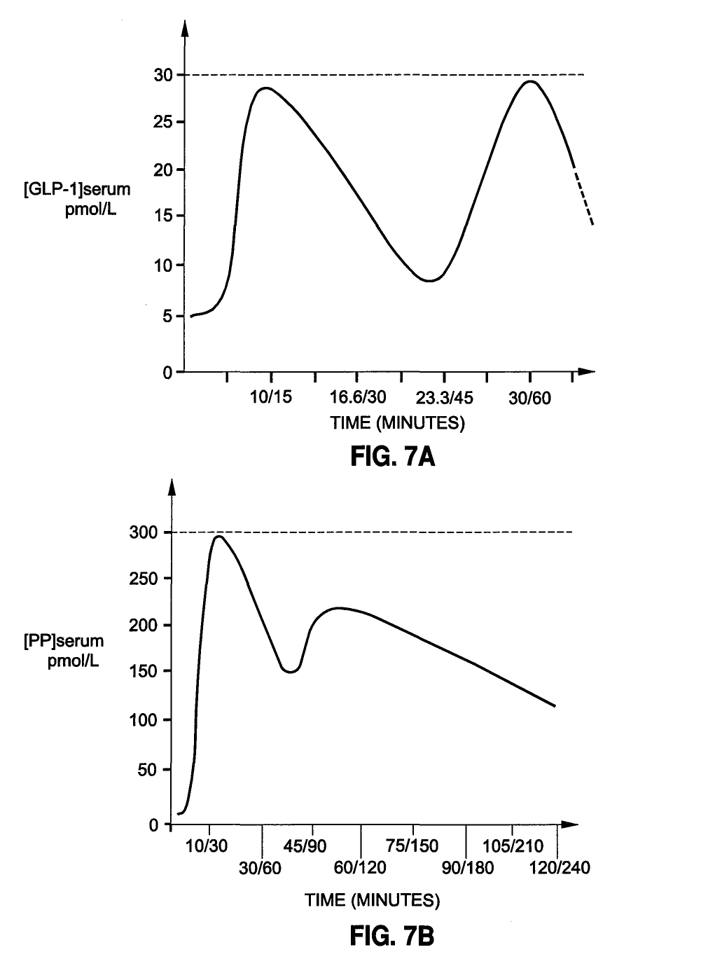

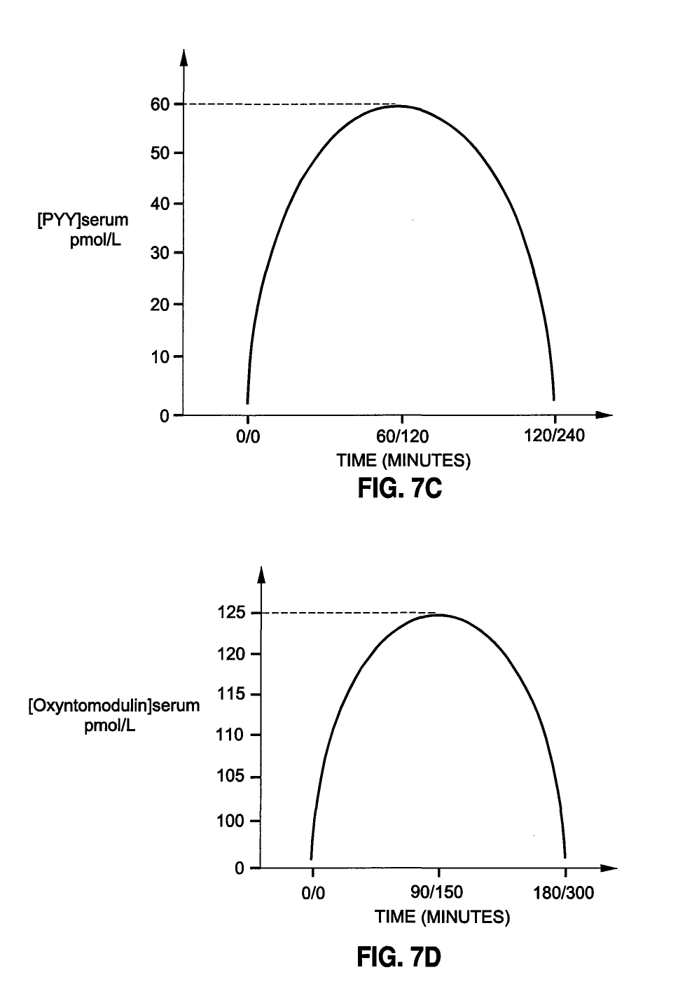

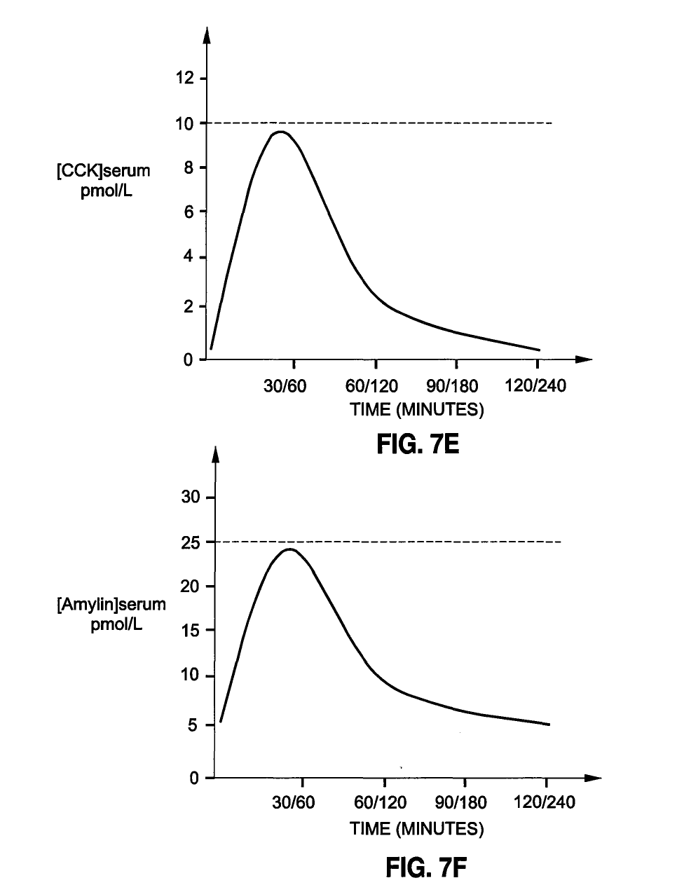

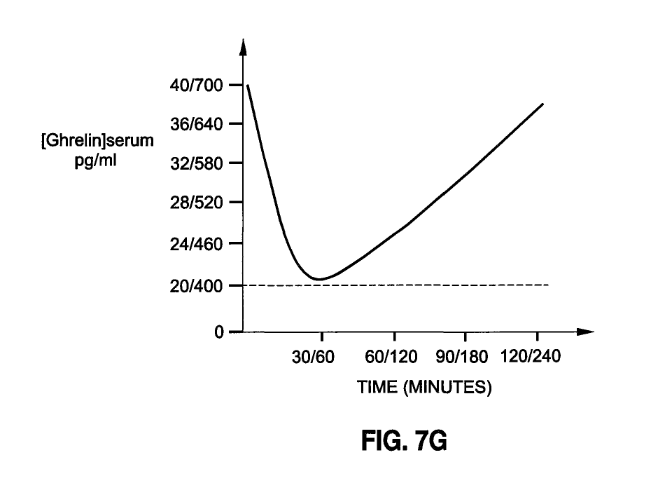

In one embodiment, a tightening of the gastric band beyond a certain threshold may trigger an acute, bimodal increase in GLP-1 production providing the patient with increased feeling of satiety be delaying gastric emptying, contributing to the ileal brake. Accordingly, in one embodiment, the hormone level for GLP-1 may be checked to ensure that an increase in GLP-1 production is, indeed, triggered by the tightening of the gastric band. If not, the gastric band may be further tightened until the acute increase is obtained. FIG. 7A illustrates an example of a model of an acute, triggered GLP-1 response. As shown, GLP-1 levels may increase to a first peak as soon as 10 minutes after the meal and the gastric band tightening, and a second peak roughly 30 minutes after the meal and the gastric band tightening.

In one embodiment, a tightening of the gastric band beyond a certain threshold may trigger an acute, bimodal increase in PP production providing the patient with increased feeling of satiety. More particularly, PP production in humans functions to decrease appetite and food intake, independently of gastric emptying. Accordingly, in one embodiment, the hormone level for PP may be checked to ensure that an increase in PP production is, indeed, triggered by the tightening of the gastric band. If not, the gastric band may be further tightened until the acute increase is obtained. FIG. 7B illustrates an example of a model of an acute, triggered PP response. As shown, PP levels may increase to a first peak around 15-20 minutes after the meal and the gastric band tightening, and a second, albeit, significantly lower peak roughly 45-60 minutes after the meal and the gastric band tightening.

In one embodiment, a tightening of the gastric band beyond a certain threshold may trigger an acute, unimodal increase in PYY production providing the patient with an increased feeling of satiety. More particularly, PYY delays gastric emptying, contributing to the ileal brake. Accordingly, in one embodiment, the hormone level for PYY may be checked to ensure that an increase in PYY production is, indeed, triggered by the tightening of the gastric band. If not, the gastric band may be further tightened until the acute increase is obtained. FIG. 7C illustrates an example of a model of an acute, triggered PYY response. As shown, PYY levels may increase to a peak as soon as an hour after the meal and the gastric band tightening.

In one embodiment, a tightening of the gastric band beyond a certain threshold may trigger an acute, unimodal increase in OXM production providing the patient with an increased feeling of satiety. More particularly, OXM lessens hunger and single-meal food intake. Accordingly, in one embodiment, the hormone level for OXM may be checked to ensure that an increase in OXM production is, indeed, triggered by the tightening of the gastric band. If not, the gastric band may be further tightened until the acute increase is obtained. FIG. 7D illustrates an example of a model of an acute, triggered OXM response. As shown, OXM levels may increase to a peak as soon as 90 minutes after the meal and the gastric band tightening.

In one embodiment, a tightening of the gastric band beyond a certain threshold may trigger an acute, unimodal increase in CCK production providing the patient with increased feeling of satiety by decreasing meal size. Accordingly, in one embodiment, the hormone level for CCK may be checked to ensure that an increase in CCK production is, indeed, triggered by the tightening of the gastric band. If not, the gastric band may be further tightened until the acute increase is obtained. FIG. 7E illustrates an example of a model of an acute, triggered CCK response. As shown, CCK levels may increase to a peak as soon as 30 minutes after the meal and the gastric band tightening.

In one embodiment, a tightening of the gastric band beyond a certain threshold may trigger an acute, unimodal increase in amylin production providing the patient with increased feeling of satiety. More particularly, amylin functions to decrease meal size and food intake. Accordingly, in one embodiment, the hormone level for amylin may be checked to ensure that an increase in amylin production is, indeed, triggered by the tightening of the gastric band. If not, the band may be further tightened until the acute increase is obtained. FIG. 7F illustrates an example of a model of an acute, triggered amylin response. As shown, amylin levels may increase to a peak slightly less than 30 minutes after the meal and the gastric band tightening.

In another embodiment, tightening of the gastric band contemporaneous with the patient eating a meal may have a direct impact on the level of ghrelin production in the patient. Ghrelin, an acylated peptide produced primarily by the stomach and proximal small intestine, functions oppositely to satiation peptides. As such, it may be a problematic hormone in fighting obesity. In other words, ghrelin increases food intake by increasing the number of meals initiated, without altering their size, and it elicits numerous appetitive feeding behaviors.

As such, it may be advantageous to decrease the amount of ghrelin production for the sake of fighting obesity. Advantageously, the tightening of the gastric band (and thus forming a smaller stoma) may trigger a decrease in the level of ghrelin in the patient. However, it should be noted that either tightening the band or eating a meal alone might not result in the desired response of decreased production of ghrelin. Similarly, mere tightening of the gastric band and the patient consuming food might also not result in the response of the desired concentration level of ghrelin. In other words, the magnitude of the tightening of the gastric band may be more important with respect to reaching the optimal decreased level of ghrelin production than mere tightening of the gastric band without regard to magnitude. Hence, by using one or more of the methods described herein, improved weight-loss efficacy may be achieved. In one embodiment, a tightening of the gastric band beyond a certain threshold may trigger an acute, reverse unimodal decrease in ghrelin production. FIG. 7G illustrates an example of a model of an acute, triggered ghrelin response. As shown, ghrelin levels may decrease to a valley at about 30 minutes after the meal and the gastric band tightening.

As shown in FIGS. 7A-7G, the x-axis labeled “time (minutes)” may include two numbers since different individuals may have different hormone response times depending on a variety of factors such as caloric load and carbohydrate/protein/fat ratio. For example, FIG. 7A should be read as “10”, “16.6”, “23.3” and “30” for a low end range (e.g., individuals with fast hormone response times) and “15”, “30”, “45” and “60” for a high end range (e.g., individuals with slower hormone response times). In other words, one individual with a fast hormone response time may release GLP-1 as quick as 10 minutes after the meal and again at 30 minutes after a meal as shown by the peaks of FIG. 7A, whereas a second individual with a slow hormone response time may release GLP-1 as slow as 15 minutes after a meal and again at 60 minutes after a meal as shown by the peaks of FIG. 7A. Various tests may be used by physicians to determine whether an individual has a slow response time or a fast response time (or somewhere in-between). Alternatively, a sensor (e.g., sensor 520 of FIG. 5) may be used to determine when a patient has the “hormone spikes” of FIG. 7A to determine if the patient has a fast hormone response time or a slow hormone response time.

With respect to FIG. 7G, describing the effects of ghrelin, the same principle described above applies to the y-axis indicting ghrelin serum levels. Ghrelin serum levels may depend on the severity of obesity of the patient and the weight loss state. As such, as shown in FIG. 7G, some individuals may decrease in ghrelin from 40 pg/ml down to a valley of 20 pg/ml while other individuals may decrease in ghrelin from 700 pg/ml to 400 pg/ml.

In another embodiment, different tightening magnitudes may correspond with optimal levels of different hormones in different individuals. In other words, the optimal level for a particular person may be unique and based on one or more other factors such as age, weight, sex, height and the like. However, as an example, for an obese person having a BMI of 30 or greater, the optimal level for GLP-1 would be slightly below 30 pmol/L as shown in FIG. 7A. So, if peak levels for GLP-1 as measured after adjustment of the gastric band for an average person is substantially below 30 pmol/L (e.g., 10-15 pmol/L), tightening of the gastric band may be needed. Conversely, a concentration level of greater than 60 pmol/L of GLP-1 may cause nausea in the patient and may require loosening of the gastric band as an attempt to lower the concentration level of GLP-1 towards the optimized levels. Similarly, FIGS. 7B-7G illustrate optimal concentration levels for the respective satiety hormones at the peaks (and valley for ghrelin). And analogous to the example discussed above with respect to GLP-1, if the peak levels for the respective satiety hormones as measured after adjustment of the gastric band for an average person is substantially below the optimal levels, tightening of the gastric band may be needed. Conversely, a concentration level significantly greater the respective peak levels may cause nausea or other discomfort in the patient and may require loosening of the gastric band as an attempt to lower the concentration level. Notably for ghrelin, as shown by FIG. 7G, the gastric band may be tightened to lower the concentration level down to the desirable valley, and the gastric band may be loosened to raise the concentration level if the ghrelin levels are significantly lower than the valley as shown in FIG. 7G.

In one example, a patient visit schedule may be used in conjunction with any of the methods described herein. For instance, a first patient-check-up visit to determine whether adjustment to the band is necessary may occur roughly 4 weeks after surgery to implant the gastric band. Follow-up visits may be scheduled every 4 to 8 weeks thereafter and may begin to taper off at 6 months, with 6 and 12 month follow-up visits thereafter. More particularly, one example of a patient visit schedule may be as follows. First visit at 4 weeks after band implant, the second visit at 8 weeks after implant, third visit at 12 weeks after implant, fourth visit at 20 weeks after implant, fifth visit at 28 weeks after implant, sixth visit at 52 weeks after implant, and an adjustment every 52 weeks thereafter.

In one embodiment, during patient visits, the sensor, if used, may be tested to determine whether the sensor is working properly. In another embodiment, the gastric band may be re-configured (if necessary) to contract and loosen based on a schedule coinciding with when the patient is usually eating in order to achieve the optimal hormone response desired (e.g., the patient reports eating at later times in the day, so the gastric band may be reconfigured to tighten to a predetermined constriction between 9:00-10:00 AM, 2:00-3:00 PM and 7:00-8:30 PM daily in order to coincide with patient meal consumption times as opposed to a previously configured constriction between 8:00-9:30 AM, 12:00-1:00 PM and 5:45-7:00 PM).

While certain hormone responses have been described herein, they are merely examples. Indeed, for any particular hormone impacting appetite and weight-control, a gastric band adjustment may result in a chronic increase, an acute increase, a chronic decrease or an acute decrease. In addition, the increase or decrease in concentration level may follow any one of the following models: a unimodal model, a bimodal model, a polimodal model, a sigmoidal model, a reverse sigmoidal modal and a logarithmic model.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references may have been made to patents and printed publications in this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of determining whether a patient is at or near optimal satiety-inducing agent concentration levels following a gastric band adjustment comprising:
collecting a bodily fluid sample from the patient in response to waiting a predetermined amount of time after the gastric band adjustment;
detecting a concentration level of one or more satiety-inducing agents from the bodily fluid sample; and
comparing the detected concentration levels of the one or more satiety-inducing agents with a predetermined range for each of the one or more satiety-inducing agents.

2. The method of claim 1, wherein the satiety-inducing agent is a hormone.

3. The method of claim 1, wherein the bodily fluid is blood.

4. The method of claim 2, wherein the hormone is one of: a peptide hormone and a brain-gut satiety hormone.

5. A method of determining whether a patient is at or near an optimal satiety-inducing agent concentration level, comprising:
collecting a first bodily fluid sample from the patient;
adjusting a gastric band;
collecting a second bodily fluid sample from the patient after adjusting the gastric band;
for at least one satiety-inducing agent, determining a difference between a satiety-inducing agent concentration level of the first bodily fluid sample and a satiety-inducing agent concentration level of the second bodily fluid sample; and
for each satiety-inducing agent concentration level difference determined, comparing the difference with a predetermined range.

6. The method of claim 5, further comprising:
performing one of tightening or loosening the gastric band in response to determining that at least one satiety-inducing agent concentration level difference is not within the predetermined range.

7. The method of claim 5, wherein the satiety-inducing agent is a hormone.

8. The method of claim 5, wherein the bodily fluid is blood.

9. A device fully insertable into a human body, configured to determine a concentration level of one or more satiety-inducing agents in plasma, the device comprising:
a processor;
a memory coupled to the processor and located within the body, the memory storing instructions, that when executed cause the device to:
collect a bodily fluid sample from the human body;
detect a concentration level of one or more satiety-inducing agents from the plasma; and
compare the detected concentration levels of the one or more satiety-inducing agents with a predetermined range for each of the one or more satiety-inducing agents.

10. The device of claim 9, wherein the memory further stores instructions that when executed, cause the device to:
transmit to a receiver, the result of comparing the detected concentration levels of the one or more satiety-inducing agents with a predetermined range for each of the one or more satiety-inducing agents.

11. The device of claim 9, wherein the satiety-inducing agent in is a hormone.

12. A device for determining a concentration level of one or more satiety-inducing agents in plasma, the device comprising:
a device body configured to be fully insertable into a human body;
a sensor connectable to the device body, configured to detect a concentration level of one or more satiety-inducing agents from plasma within the human body;
a storage configured to store a predetermined range corresponding to each of the one or more satiety-inducing agents;
a comparator configured to compare the concentration level of the one or more satiety-inducing agents detected by the sensor to the predetermined range corresponding to each of the one or more satiety-inducing agents; and
a transceiver configured to communicate the output of the comparator to a receiver.

13. The device of claim 12, wherein each of the satiety-inducing agents is a different hormone.

14. A device fully insertable into a human body, configured to determine a concentration level of one or more satiety-inducing agents in plasma and further configured to communicate with a gastric band attached to the human body, the device comprising:
a processor;
a memory coupled to the processor and located within the body, the memory storing instructions, that when executed cause the device to:
detect a concentration level of one or more satiety-inducing agents from the plasma;
determine that the concentration levels of the one or more satiety-inducing agents detected from the plasma is outside a predetermined range; and
transmit a command to adjust a constriction level of a gastric band in response to determining that the concentration levels of the one or more satiety-inducing agents detected from the plasma is outside the predetermined range.

15. The device of claim 14, wherein each of the one or more satiety-inducing agents is a hormone.

16. The device of claim 14, wherein in response to the command to adjust the constriction level of the gastric band, the concentration level of at least one satiety-inducing agent increases or decreases acutely.

17. The device of claim 14, wherein in response to the command to adjust the constriction level of the gastric band, the concentration level of at least one satiety-inducing agent increases or decreases in accordance with a bimodal model.

18. The device of claim 14, wherein in response to the command to adjust the constriction level of the gastric band, the concentration level of at least one satiety-inducing agent increases or decreases in accordance with a polimodal model.

19. The device of claim 14, wherein in response to the command to adjust the constriction level of the gastric band, the concentration level of at least one satiety-inducing agent increases or decreases in accordance with a uni-modal model.

20. The device of claim 14, wherein in response to the command to adjust the constriction level of the gastric band, the concentration level of at least one satiety-inducing agent increases or decreases in accordance with a logarithmic model.

21. A method for determining an optimal amount of constriction applied by a gastric band implanted around a stomach of an obese patient, the method comprising the steps of:
laparoscopically implanting the gastric band around the stomach of an obese patient;
collecting a blood, serum or plasma sample from the obese patient;
detecting a concentration level of one or more satiety-inducing agents in the sample; and
if the concentration level is higher than optimal, adjusting the gastric band so that it applies a lesser constriction to the stomach, and
if the concentration level is lower than optimal, adjusting the gastric band so that it applies a greater constriction to the stomach.
